(19) United States
(12) Patent Application Publication
(10) Pub. No.: US 2014/0268039 A1
(43) Pub. Date: Sep. 18, 2014

(54) SYSTEMS AND METHODS FOR VARIABLE MODE OPTICAL COHERENCE TOMOGRAPHY IMAGING

(71) Applicant: CARL ZEISS MEDITEC, INC., Dublin, CA (US)

(72) Inventors: Kabir M. ARIANTA, Livermore, CA (US); Homayoun BAGHERINIA, Oakland, CA (US); Tilman SCHMOLL, Dublin, CA (US); Alexandre R. TUMLINSON, San Leandro, CA (US); Yingjian WANG, Fremont, CA (US)

(73) Assignee: CARL ZEISS MEDITEC, INC., Dublin, CA (US)

(21) Appl. No.: 13/803,522

(22) Filed: Mar. 14, 2013

(51) Int. Cl. A61B 3/14

(52) U.S. Cl. CPC A61B 3/14
USPC 351/206

(57) ABSTRACT

Systems and methods for improvements to optical coherence tomography systems for operating in different imaging modes are presented. In one embodiment, a system for identifying the presence and type of an adjunct lens operably connected to the OCT instrument for changing between imaging modes in the system is described. In a second embodiment, a system for dynamically autofocusing the OCT system depending on the layer of interest is presented. In a third embodiment, the overall power of the system used for imaging can be adjusted depending on the location and type of scan desired while accounting for the safety standards for recommended light exposure.

### TECHNICAL FIELD

The present invention relates to the field of ophthalmic imaging, and in particular optical coherence tomography imaging systems.

### BACKGROUND

Optical Coherence Tomography (OCT) is a technique for performing high-resolution cross-sectional imaging that can provide images of tissue structure on the micron scale in situ and in real time (Huang et al. “Optical Coherence Tomography” Science 254(5035):1178 1991). OCT is a method of interferometry that determines the scattering profile of a sample along the OCT beam. Each scattering profile is called an axial scan, or A-scan. Cross-sectional images (B-scans), and by extension 3D volumes, are built up from many A-scans, with the OCT beam moved to a set of transverse locations on the sample. OCT provides a mechanism for micrometer resolution measurements.

In frequency domain OCT (FD-OCT), the interferometric signal between light from a reference and the back-scattered light from a sample point is recorded in the frequency domain rather than the time domain. After a wavelength calibration, a one-dimensional Fourier transform is taken to obtain an A-line spatial distribution of the object scattering potential. The spectral information discrimination in FD-OCT is typically accomplished by using a dispersive spectrometer in the detection arm in the case of spectral-domain OCT (SD-OCT) or rapidly scanning a swept laser source in the case of swept-source OCT (SS-OCT).

Evaluation of biological materials using OCT was first disclosed in the early 1990's (see for example U.S. Pat. No. 5,321,501 hereby incorporated by reference). Frequency domain OCT techniques have been applied to living samples (see for example Nassif et al. “In vivo human retinal imaging by ultrahigh-speed spectral domain optical coherence tomography” Optics Letters 29(5):480 2004). The frequency domain techniques have significant advantages in speed and signal-to-noise ratio as compared to time domain OCT (see for example Choma, M. A., et al. “Sensitivity advantage of swept source and Fourier domain optical coherence tomography” Optics Express 11(18): 2183 2003). The greater speed of modern OCT systems allows the acquisition of larger data sets, including 3D volume images of human tissue.

OCT technology has found widespread use in ophthalmology for imaging different areas of the eye and providing information on various disease states and conditions. Commercial OCT devices have been developed for imaging both the anterior and posterior sections of the eye (see for example Cirrus HD-OCT, Visante Omni, and Stratus (Carl Zeiss Meditec, Inc. Dublin, Calif.)). The Cirrus HD-OCT system allows for imaging both the anterior and posterior regions by inserting a lens to change the focal properties of the system as described in US Patent Publication No. 2007/0291277. In addition to collecting data at different depth locations, different scan patterns covering different transverse extents can be desired depending on the particular application. It is an object of the present invention to provide improvements to OCT systems for changing between modes to allow for efficient, safe, and convenient OCT imaging of the eye.

### SUMMARY

Aspects of the present invention are directed towards improvements to ophthalmic OCT systems capable of imaging different locations or different disease states in the eye. In one embodiment, a system for identifying the presence and type of an adjunct lens operably connected to the OCT instrument for changing between imaging modes in the system is described. In a second embodiment, a system for dynamically autofocusing the OCT system depending on the layer of interest is presented. In a third embodiment, the overall power of the system used for imaging can be adjusted depending on the location and type of scan desired while accounting for the safety standards for recommended light exposure. All three embodiments allow for more efficient, safe, and increased convenience for the instrument operator in OCT ophthalmic imaging.

### BRIEF DESCRIPTION OF THE DRAWINGS

FIG. 1 shows a generalized diagram of an FD-OCT system that could be used with the present invention.

FIG. 2 illustrates an OCT system used in conjunction with an iris viewing and line scanning opthalmoscope to provide different views of the eyes during imaging.

FIG. 3 shows the elements involved with a preferred embodiment of the present invention directed towards detecting the presence and identity of an add-on lens module to an OCT system.

FIGS. 4a and 4b show images collected on the iris camera of an OCT system when two different external lens modules were attached to the instrument. FIG. 4c shows an image collected on the iris camera of an OCT system when the external lens module was not attached.

FIG. 5 shows an image of the information of interest for external lens module detection in a preferred embodiment of the invention that is isolated from the image shown in FIG. 4a.

FIG. 6 illustrates vertical profiles identified in the image using information on the centroids of the information of interest according to one embodiment of the present invention.

FIG. 7a shows three vertical profiles generated from the image shown in FIG. 4a. FIG. 7b shows three vertical profiles generated from the image shown in FIG. 4b. FIG. 7c shows three templates for the images shown in FIG. 4 that can be used for template matching to identify external lens modules according to one aspect of the present invention.

FIG. 8 illustrates the steps involved with an embodiment of the present invention in which the system dynamically autofocuses to a specific tissue in the eye.

### DETAILED DESCRIPTION

A generalized FD-OCT system used to collect 3-D image data suitable for use with the present invention is illustrated in FIG. 1. A FD-OCT system includes a light source 101, typical sources including but not limited to broadband light sources with short temporal coherence lengths or swept laser sources. (See for example, Wojtkowski, et al., “Three-dimensional retinal imaging with high-speed ultrahigh-resolution optical coherence tomography,” Ophthalmology 112(10):1734 2005 or Lee et al. “In vivo optical frequency domain imaging of human retina and choroid,” Optics Express 14(10):4403 2006 the contents of both of which is hereby incorporated by reference)

Light from source 101 is routed, typically by optical fiber 105, to illuminate the sample 110, a typical sample being tissues at the back of the human eye. The light is scanned, typically with a scanner 107 between the output of the fiber and the sample, so that the beam of light (dashed line 108) is scanned over the area or volume to be imaged. Light scattered from the sample is collected, typically into the same fiber 105 used to route the light for illumination. Reference light derived from the same source 101 travels a separate path, in this case involving fiber 103 and retro-reflector 104. Those skilled in the art recognize that a transmissive reference path can also be used. Collected sample light is combined with reference light, typically in a fiber coupler 102, to form light interference in a detector 120. The output from the detector is supplied to a processor 130. The results can be stored in the processor or displayed on display 140. The processing and storing functions may be localized within the OCT instrument or functions may be performed on an external processing unit to which the collected data is transferred. This unit could be dedicated to data processing or perform other tasks which are quite general and not dedicated to the OCT device.

The interference causes the intensity of the interfered light to vary across the spectrum. The Fourier transform of the interference light reveals the profile of scattering intensities at different path lengths, and therefore scattering as a function of depth (z-direction) in the sample (see for example Leitgeb et al. “Ultrahigh resolution Fourier domain optical coherence tomography,” Optics Express 12(10):2156 (2004)). The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube.

The sample and reference arms in the interferometer could consist of bulk-optics, fiber-optics or hybrid bulk-optic systems and could have different architectures such as Michelson, Mach-Zehnder or common-path based designs as would be known by those skilled in the art. Light beam as used herein should be interpreted as any carefully directed light path. In time-domain systems, the reference arm needs to have a tunable optical delay to generate interference. Balanced detection systems are typically used in TD-OCT and SS-OCT systems, while spectrometers are used at the detection port for SD-OCT systems. The invention described herein could be applied to any type of OCT system. The system is typically enclosed in a housing with various patient positioning components including chin and headrest.

OCT systems can have additional imaging modalities incorporated into the system to aid in alignment or provide additional clinical information. One example of such a system is illustrated in FIG. 2 and described in detail in US Patent Application US 2007/0291277 hereby incorporated by reference and realized in the commercially available product Cirrus HD-OCT (Carl Zeiss Meditec, Inc. Dublin, Calif.). In this system, an iris viewer and a line scanning opthalmoscope (LSO) are operated in conjunction with an OCT system. The LSO provides a view of the fundus in parallel to the OCT information. The iris camera provides a view of the iris of the patient's eye and is used primarily to align the patient's eye with the optical axis of the device. The iris viewer includes an LED for illuminating the eye typically positioned close to the ocular lens 201. Preferably, the LED generates light having a wavelength of about 700 nm. The reflected 700 nm light is captured by lens 201 and travels back through splitter BS1 to splitter BS2 where it is reflected back through a series of lenses to a CMOS camera. The camera generates an output which is supplied to a monitor that will display an image of the iris on a user interface of the device.

### External Corneal Lens Module Detection

In a first embodiment of the present invention, the OCT system is equipped with a system to detect the presence and identify the type of an add-on external lens that can be attached to the outside of the instrument to enable to system to operate in different imaging modes. In a preferred embodiment of the present invention, the external lens module is used for anterior segment scanning of human eye using an OCT instrument. In the preferred embodiment it is possible to detect the presence of an add-on external lens module on the ocular housing of an OCT instrument without using electrical or electro-mechanical sensors. It can be accomplished using optical components of the iris camera. This is advantageous in that it can allow upgrades to existing commercial systems without extensive hardware rework, only new software and the desired lens module need to be installed in the field.

FIG. 3 illustrates a preferred embodiment for detecting the presence and identifying add-on lenses to an ophthalmic OCT system. A section of the instrument housing (labeled ocular housing 310) holding the ocular lens 312 in place is illustrated. One or more lenses are mounted on an injection molded housing or fixture 314 (add-on lens and housing) and attached to the ocular housing, preferably using magnets though other means of attachment could be envisioned by those skilled in the art. In practice, one type of add-on lens could be used for pachymetry measurements and narrows the field of view. Alternatively, sometimes a field of view wider than is available with the primary lens 312 is desired and a different add-on lens can provide that wider field of view.

In a preferred embodiment, part of one or more of the add-on lenses is provided with a small diffused feature on its outer rim. The diffused feature can be created by painting a small part or area of the lens white or gray. Alternatively, a small area of the injection molded housing where the lens is held in place can be painted. Light from light source 318 in the ocular housing used for the iris camera 320 as described in reference to FIG. 2 illuminates the diffused feature on or behind the lens and is imaged through a light mask 324 onto the instrument's viewing camera.

The mask 324 is provided with transparent regions that provide a code indicative of the type of add-on lens being used. In a most basic example, where there are two different types of add-on lenses, the mask associated with one type of lens could have a single opening and the mask associated with the second type of lens could have two openings. The diffused light pattern transmitted by these openings is imaged by camera 320. The image is analyzed using an algorithm as will be described in further detail below to indicate the presence and type of the add-on external lens module on the instrument's ocular housing. Different patterns of diffuse light could be used to identify different lens elements and in response thereto the system could make automatic adjustments with account for the change in the field of view or depth. Such changes could include adjusting the length of the reference arm or inserting or removing a “flip-in” lens in the sample arm path located within the instrument housing.

In a preferred embodiment, a prism 330 can be mounted on the module's housing for illuminating the patient's eye when the add-on module is placed on the instrument's ocular housing. The prism helps correct for the fact that module changes the working distance to the eye so that the light from source 318 would not be properly directed to the eye absent the prism.

An algorithm that can be used to detect and identify the add-on lens will now be described in further detail. FIGS. 4a and 4b show two images collected on the ophthalmic system's iris camera with two different types of external lens modules attached to the instrument. Two small spots of light (410, 412) can be seen at the bottom of the image in FIG. 4a. This would correspond to a mask having two openings transmitting the diffuse light. In contrast, in FIG. 4b, one small spot 414 can be seen at the bottom of the image, corresponding to a mask having one opening transmitting the diffuse light. An image collected without the add-on lens module attached is illustrated in FIG. 4c for comparison.

The spots can be isolated from the image using various imaging processing techniques as would be appreciated by someone skilled in the art. Here connected component analysis is used to generate an image as shown in FIG. 5. Two distinct spots of light are evident. The centroids of the spots can be identified and used to generate vertical profiles in the image as shown in FIG. 6. The lines (610, 612, 614) are drawn through regions of maximum and minimum intensity in the image. The vertical intensity profiles of these lines are plotted as shown in FIG. 7a and cross-correlations between these vertical profiles can be analyzed. The normalized cross correlations between two of the profiles (610, 614) is much higher (0.9615) than the others (0.2909 and 0.0326) indicating two relatively strong “peaks”. This is used to indicate the presence of the add-on lens with two distinct spots. In comparison the cross-correlations for the image taken without the external lens module are shown in FIG. 7b.

In an alternative embodiment of the present invention, template matching could be also used to detect the spots. A 2-D normalized cross-correlation function between the image and each template as shown in FIG. 7c can indicate the location of the templates. It is assumed that the amount of rotation, scaling or shear (affine transform) is not significant to affect the performance of 2-D normalized cross-correlation. The resulting output of the 2-D normalized cross-correlation function is a 2-D map λ(u,v), which can range from −1 and 1. The position (x,y) of the maximum value γₘₐₓ in this map represents the center location of the template within the image. The type and presence of the add-on lens is based on the number and locations of the spots with high γₘₐₓ values (e.g. >0.9). Here the cross correlations are relatively equal (or high) and indicate the absence of the external lens. As mentioned above, various patterns could be used for different lenses and more complex algorithms could be used to identify different external lens modules and imaging properties such as reference arm adjustment, scan type, and scan geometry could be automatically adjusted on the instrument based on the identification.

While the preferred embodiment utilized light from the iris camera, it would be possible to use a other light sources and cameras that may be present in the system as would be appreciated by someone skilled in the art.

### Method to Autofocus to a Specific Layer

In optical coherence tomography, the best signal to noise ratio and lateral resolution are achieved when the beam is focused to a small spot within the object of interest. In ophthalmic OCT, the position of the beam focus is affected by the subjects own optics which vary widely from person to person. Therefore it is typical to adjust the optical properties of the OCT device to bring the desired tissue into focus such that an acceptable image is acquired. In the past, OCT focus has been set in a number of ways including but not limited to: manually focusing to a criteria where the operator found the image most acceptable, systematically stepping through focal positions to build up a tomogram consisting of many well focused layers, simultaneously acquiring tomograms at multiple focal positions, automatically optimizing some external signal, for example maximizing the signal returned from a simultaneously acquired confocal scanning laser opthalmoscope with a known focal position relative to the OCT, automatically optimizing a global parameter over the entire OCT image such as maximizing image entropy, or peak signal intensity, and automatically optimizing a parameter such as entropy or peak signal for a particular tissue layer of interest.

A second embodiment of the present invention increases the signal to noise ratio and lateral resolution of an optical coherence tomogram at a specified tissue layer. It does this by applying an offset to a focal position found by a traditional autofocus algorithm. The present solution is superior to previous solutions because it allows for focus position to be optimized for a particular tissue layer of interest, that may have weak or otherwise unsuitable signal for determining focus, in a way that takes advantage of the relative ease of finding a nearby layer.

The system first automatically finds focus of an easily identified layer using either an OCT beam or a probe beam of an alternate modality such as confocal imaging. A traditional algorithm may be able to precisely locate some features of a tissue such as by maximizing signal at a tissue boundary or at a tissue location known to have particularly high signal or contrast of some sort. For example, the retinal pigment epithelium (RPE) often provides such a bright, high contrast layer in the posterior eye. The actual layer of interest may be more difficult to find by such an algorithm because it has low signal or contrast. The choroid and vitreous are examples of such layers.

The system dynamically determines an offset to the tissue of interest—either by examining preliminary OCT data, or by applying a known offset for a particular tissue type. This determining of offset maybe performed before or after best focus is found at the ‘easy layer’.

The distance offset to the actual layer of interest may be known or estimated a priori, or the offset may be measured from properties of the optical coherence tomogram. When the offset is determined, the optical path of the system can be adjusted to move the focus to provide best signal at the offset layer. The device may always optimize to the same tissue layer as determined dynamically from OCT data.

The system applies the offset to the OCT beam away from the optimum focus at the easy layer. This may happen sequentially after first finding the easy layer and then stepping away, or may occur simultaneously such that the OCT beam focused on the layer of interest is shifted away from a simultaneously acquired beam that remains focused on the easy layer. The latter method would allow for focus tracking if focus might change, such as by eye accommodation. The operator may select to have the view optimized by the device for a particular tissue from a list of possible tissue layers, or at a specified offset from a particular tissue layer. The operator may select a particular analysis from the device and the device will select to acquire tomograms at the focus position or positions best suited to make the analysis, and those analyses would then be provided to the instrument user.

Some image processing methods, such as interferometric synthetic aperture microscopy (see for example Ralston et al. “Interferometric synthetic aperture microscopy” Nature Physics 3, 129-134 2007) may actually have advantages when used with the OCT beam slightly out of focus at the tissue layer of interest, such that the information from a single location is spread to multiple measurements which can later be combined. In such a case, the above method could be used to apply an optimal focus, which is itself at an offset from the particular layer of interest. In this case the layer of interest could be the easily found layer.

### Dynamic Power Adjustment

In a further embodiment of the present invention, the system is able to dynamically adjust the optical output power of an OCT system. Commonly the optical output power of an OCT system is adjusted to a fixed optical output power which is below the worst case maximum permissible exposure (MPE) value of all the imaging modes of the device. However, different imaging modes could benefit from different optical output powers. There are cases where the power may be reduced, for example during alignment, in case sufficient image quality is achieved also with lower power. Or when highly reflective samples are scanned, which may cause the detector to saturate when imaged with too much sample power (e.g. the central part of the cornea). And there are other cases where the image quality is unsatisfactory while the optical output power is significantly below the MPE value for the current scan mode, for example for extremely short scans, where the probing beam is scanned very quickly over a large area on the retina. In such cases the device may use its image quality information of the alignment scans in order to set the optical output power for the acquisition. Since higher optical power incident on the sample directly results in better system sensitivity, the image quality could be significantly improved.

In an alternative embodiment, the device could adjust the optical output power in general to the MPE for the current imaging mode. This would ensure maximum sensitivity for each imaging mode, rather than limiting the system's output power and therefore system sensitivity according to the worst case imaging mode. In one case, the mode could depend on the location of the tissue being imaged. The MPE for an anterior segment imaging mode is in general higher than the MPE for a posterior segment imaging mode so the system could be configured to image at higher powers for scans in the anterior segment.

Although various applications and embodiments that incorporate the teachings of the present invention have been shown and described in detail herein, those skilled in the art can readily devise other varied embodiments that still incorporate these teachings. Although the description of the present invention is discussed herein with respect to the sample being a human eye, the applications of this invention are not limited to eye and can be applied to any application using OCT.

The following references are hereby incorporated by reference:

### Patent Documents

U.S. Pat. No. 5,321,501
U.S. Pat. No. 6,095,648
US Patent Publication No. 2007/0291277
US Patent Publication No. 2008/0106696

### Non-Patent Literature

Huang et al. “Optical Coherence Tomography” Science 254(5035):1178 1991.
Nassif et al. “In vivo human retinal imaging by ultrahigh-speed spectral domain optical coherence tomography” Optics Letters 29(5):480 2004.
Choma, M. A., et al. “Sensitivity advantage of swept source and Fourier domain optical coherence tomography” Optics Express 11(18): 2183 2003.
Hee et al., “Optical Coherence Tomography for Ophthalmic Imaging,” IEE Engineering in Medicine and Biology, January/February 1995.
Wojtkowski, et al., “Three-dimensional retinal imaging with high-speed ultrahigh-resolution optical coherence tomography,” Ophthalmology 112(10):1734 2005.
Lee et al. “In vivo optical frequency domain imaging of human retina and choroid,” Optics Express 14(10):4403 2006.
Leitgeb et al. “Ultrahigh resolution Fourier domain optical coherence tomography,” Optics Express 12(10):2156 (2004).
Ralston et al. “Interferometric synthetic aperture microscopy” Nature Physics 3, 129-134 2007.

What is claimed is:

1. An optical coherence tomography (OCT) system for generating images of the eye and capable of switching between different imaging modes, said system comprising;
a light source for generating a light beam propagating along an axis;
a beam divider for directing a first portion of the light beam into a reference arm and a second portion of the light beam into a sample arm;
optics for scanning the light beam in the sample arm over the eye of a patient to a plurality of positions in a plane perpendicular to the propagation axis of the beam;
a detector for measuring light radiation returning from the sample and reference arms and generating output signals in response thereto;
a processor for generating images based on the output signals;
a first lens for focusing the light beam;
a housing for containing the source, beam divider, detector and first lens;
a second lens operably attached to the housing for changing the focusing properties of the first lens; and
means for detecting the presence and/or type of the second lens attached to the housing.

2. An OCT system as recited in claim 1 wherein said means detects an optical signal associated with the second lens.

3. An OCT system as recited in claim 1, wherein said means includes a camera for identifying the presence and/or type of second lens attached to the housing.

4. An OCT system as recited in claim 3, wherein the second lens has a diffuse region outside the central aperture of the lens.

5. An OCT system as recited in claim 4 further including a mask located between second lens and the camera, said mask being coded with openings for filtering the diffuse light and identifying the second lens.

6. An OCT system as recited in claim 5, wherein cross correlation between features in an image generated by the camera are used to identify the presence of the second lens.

7. An optical coherence tomography (OCT) system for generating images of the eye said system comprising;
a light source for generating a light beam propagating along an axis;
a beam divider for directing a first portion of the light beam into a reference arm and a second portion of the light beam into a sample arm;
optics for scanning the light beam in the sample arm over the eye of a patient to a plurality of positions in a plane perpendicular to the propagation axis of the beam and for focusing the system to a specific depth in the eye;
a detector for measuring light radiation returning from the sample and reference arms and generating output signals in response thereto; and
a processor for generating images based on the output signals, said processor for further identifying a particular layer based on a quality of the image, and for dynamically adjusting the optics using an offset from the determined layer to change the focus of the system.

8. An OCT system as recited in claim 7, wherein the offset is determined based on a priori knowledge of the physiology of the eye.

9. An OCT system as recited in claim 7, wherein the offset is determined using properties of the generated images.

10. An optical coherence tomography (OCT) system for generating images of the eye said system comprising;
a light source for generating a light beam propagating along an axis;
a beam divider for directing a first portion of the light beam into a reference arm and a second portion of the light beam into a sample arm;
optics for scanning the light beam in the sample arm over the eye of a patient to a plurality of positions in a plane perpendicular to the propagation axis of the beam;
a detector for measuring light radiation returning from the sample and reference arms and generating output signals in response thereto; and
a processor for generating images based on the output signals and for analyzing the quality of the images and for adjusting the power of the light source based on information on the quality of the image.

11. An OCT system as recited in claim 10, wherein the source power is decreased in an alignment mode and increased in an acquisition mode.

12. An optical coherence tomography (OCT) system for generating images of the eye said system comprising;
a light source for generating a light beam propagating along an axis;
a beam divider for directing a first portion of the light beam into a reference arm and a second portion of the light beam into a sample arm;
optics for scanning the light beam in the sample arm over the eye of a patient to a plurality of positions in a plane perpendicular to the propagation axis of the beam;
a detector for measuring light radiation returning from the sample and reference arms and generating output signals in response thereto; and
a processor for generating images based on the output signals; and
a user interface for selecting a plurality of imaging modes and wherein the processor adjusts the power of the light source based on the imaging mode selected by the user.

13. An OCT system as recited in claim 12, wherein the power is adjusted based on the maximum permissible exposure determined for a particular imaging mode.

14. An optical coherence tomography (OCT) system, said system having a housing, and, mounted with the housing, is a light source for generating a beam of radiation, a beam divider for dividing the beam along sample and reference arms, and a detector for monitoring the light received from both the sample and reference arms and a processor for generating images based on signals generated by the detector, with said sample arm terminating in a primary lens, said system further including an imaging camera, separate from the detector for generating an image of the light passing through the main lens, said system comprising:
a fixture mountable on the housing in alignment with the primary lens, said fixture carrying at least one secondary lens for changing the field of view of the system, said fixture including on optical reference for identifying the secondary lens, said optical reference being imaged by the camera.

15. A system as recited in claim 14 wherein image generated by the camera is used by the processor to modify the generation of the images based on the type of secondary lens in the fixture.

16. A system as recited in claim 14 wherein the optical reference comprises a region for creating diffuse light and a coded mask having one or more regions of transparency for transmitting diffuse light to the camera.

17. An OCT system as recited in claim 14, wherein cross correlation between features in the image generated by the camera are used to identify the presence of the second lens.
